(19) United States
(12) Patent Application Publication
(10) Pub. No.: US 2024/0038393 A1
(43) Pub. Date: Feb. 1, 2024

(54) PREDICTING DISEASE PROGRESSION BASED ON DIGITAL-PATHOLOGY AND GENE-EXPRESSION DATA

(71) Applicants: GENENTECH, INC., South San Francisco, CA (US); HOFFMANN-LA ROCHE INC., Little Falls, NJ (US)

(72) Inventors: Xiao Li, Foster City, CA (US); Marius Rene Garmhausen, Grenzach-Wyhlen (DE); Gunther Jansen, Lorrach (DE); Teresa Melanie Karrer, Basel (CH)

(73) Assignees: GENENTECH, INC., South San Francisco, CA (US); HOFFMANN-LA ROCHE INC., (omitted)

(21) Appl. No.: 18/449,632

(22) Filed: Aug. 14, 2023

Related U.S. Application Data
(63) Continuation of application No. PCT/US2022/015017, filed on Feb. 3, 2022.
(60) Provisional application No. 63/149,698, filed on Feb. 16, 2021.

(51) Int. Cl. G16H 50/20; G16H 50/30; G16H 30/40; G16B 25/10; G16B 40/20

(52) U.S. Cl. CPC G16H 50/20; G16H 50/30; G16H 30/40; G16B 25/10; G16B 40/20

(57) ABSTRACT

In some embodiments, a current state of a medical condition or a progression of the medical condition is predicted by processing one or more digital pathology images and expression levels of genes using a machine-learning model. In some embodiments, one or more predicted gene-expression levels are generated by processing a data set corresponding to one or more digital pathology images using a machine-learning model. In some embodiments, one or more predicted digital pathology metrics are generated by processing a data set that corresponds to expression levels of a set of genes using a machine-learning model.

### CROSS-REFERENCE TO RELATED APPLICATIONS

This application is a continuation of International Application No. PCT/US2022/015017, filed on Feb. 3, 2022, which claims the benefit of and the priority to U.S. Provisional Application No. 63/149,698, filed on Feb. 16, 2021 and entitled “Predicting Disease Progression based on Digital-Pathology and Gene-Expression Data”, which are hereby incorporated by reference in their entireties for all purposes.

### BACKGROUND

Generating accurate predictions as to how a particular subject's medical condition (e.g., disease) will progress (e.g., if no treatment is provided or if a particular treatment is provided) would provide high value and support shaping of treatment decisions. For example, an accurate prediction as to how a particular subject's cancer will respond to a given treatment may inform whether to provide or recommend the particular subject with the given treatment. However, generating accurate predictions is particularly challenging given a high degree in heterogeneity and further by dependencies across variables.

Heterogeneity can include heterogeneity across subjects, across medical condition presentations, tumors, or across regions within a single tumor. Heterogeneity can include (for example): genetic heterogeneity (e.g., corresponding to different mutations in different subclones of the tumor); expression heterogeneity (corresponding to different expression profiles); or tumor-microenvironment heterogeneity (e.g., corresponding to different oxygen availability and nutrient availability). Heterogeneity across regions within a single tumor presents additional challenges in that a biopsy may then include multiple regions with distinct characteristics, making it all the more challenging to predict the present or potential behavior of cells and, by extension, of the tumor and disease.

Heterogeneity can also pertain to immune cells. For example, whether immune cells are present near or in a tumor can vary across subjects and/or across tumors. Even if immune cells are near a tumor, gene expression in the immune cells may vary across subjects, tumors, or regions.

Not only does heterogeneity make it more difficult to identify predictors of progression, but it also can make it more difficult to collect and/or process a data set for an individual subject that is interpretable. For example, it may be difficult to accurately predict progression when a a biopsy included heterogeneous regions or when a biopsy included only one homogeneous part of a heterogeneous tumor (thereby masking the tumor's heterogeneity and making any resulting prediction unrepresentative of the tumor as a whole).

Further, biological data sets are often large. Gene-expression data can include up to tens of thousands of expression levels, as the human genome includes approximately 30,000 genes. Given the size of this data set and the fact that expression variables are numeric values, it can be difficult to accurately predict how a particular expression level of a particular gene will influence disease progression or treatment response, much less predicting a progression or treatment response based on expression variables for multiple genes. The complexity only further explodes if multiple data types are processed.

Thus, frequently, static rules or subjective human assessments are used to select a particular portion of a data set that is available or that is potentially available, and a prediction of a disease progression or treatment efficacy is frequently made subjectively based on this data-set portion. Static rules, however, may fail to capture complexities of signals, where a given signal may materialize in different ways. For example, a defective DNA repair pathway may be caused by mutations in different genes cooperating in enabling the pathway's function. It would be advantageous to be able to objectively identify signals within large data sets that provide information that can be used to accurately predict a disease state or progression.

### SUMMARY

Techniques disclosed herein relate to predicting a medical-condition state or progression of one or more subjects based on integration of different types of data (e.g., collected using different types of techniques). The medical-condition state may identify a particular disease, disease type (e.g., type of cancer), or stage of a disease. The progression may correspond to a progression predicted for the subject(s) if no treatment for the medical condition is received or if a particular type of treatment for the medical condition is received. The predicted medical-condition progression may include (for example) a probability of survival at a particular time point (e.g., relative to data collection or treatment initiation) or a predicted amount of disease progression within a predefined time window (e.g., corresponding to a disease stage, change of a tumor-size metric). The medical condition may include cancer or a particular type of cancer. The different types of data used to predict the medical-condition state or progression may include (for example) two or more of: digital pathology data, gene-expression data, and radiology data (e.g., clinical imaging scans, CT scans, or PET data).

In some instances, an integrated processing workflow is performed, where a first technique is used to process data of a first type (e.g., gene-expression data) and to select a first set of features; a second technique is used to process data of a second type (e.g., digital pathology data) and to select a second set of features; and a third technique is used to generate the predicted result based on values of the first and second sets of features. One, more, or all of the first, second, and third techniques may use a machine-learning model (e.g., configured to shrink a feature set or process features). For example, a gene-expression data set may include expression data for each of tens of thousands of genes, and a variable-focus machine-learning model may process gene-expression data sets to identify a subset of the genes for which expression signals are predictive of or informative of a disease progression. Another data integration machine-learning model may be trained to receive expression data for the subset of genes and to also receive metrics characterizing spatial characteristics of digital pathology images (e.g., that represent absolute or relative quantities of a cell type, spatial clustering of a cell type, and/or spatial dispersion of multiple cell types) and to predict a result representative of a subject's medical-condition progression. The data integration machine-learning model and the feature-focus machine-learning model may have the same architecture or different architectures. The metrics characterizing spatial characteristics of digital pathology images may have been a predefined variable set or selected using a feature-focus machine-learning model. Thus, in some instances, each of two or more feature-focus machine-learning models is trained to shrink a feature set, and an integrating level machine-learning model is trained and configured to transform values of the shrunk feature sets into a result (e.g., predicting a disease state or progression). In some other instances, a single feature-focus machine-learning model is trained to shrink a feature set, and a data integration machine-learning model is trained to transform values of the shrunk feature set (e.g., associated with a first data type) and other values (e.g., associated with a second data type) into a result.

In some instances, a combinatorial processing workflow is performed, where a first technique is used to process data of a first type (e.g., gene-expression data) and to generate a first predicted result that predicts a subject's medical-condition progression or state. A second technique is used to process data of a second type (e.g., digital pathology data) and to generate a second predicted result that predicts the subject's medical-condition progression or state. A combinatorial machine-learning model is trained and configured to receive the first predicted result and the second predicted result and to output a transformed predicted result that predicts the subject's medical-condition progression or state.

In some instances, a hybrid processing workflow is performed, where a first technique is trained and configured to process a first set of variables corresponding to data of a first type (e.g., gene-expression data) and to identify a subset of the first set of variables. A second technique is trained and configured to process a second set of variable values corresponding to data of a second type (e.g., digital pathology data) to generate a preliminary predicted result predicting a subject's medical-condition state or progression. A hybrid machine-learning model is trained and configured to receive values for the first set of variables (e.g., values for a shrunk variable set) and the preliminary predicted result and to generate a transformed predicted result predicting the subject's medical-condition state or progression.

To illustrate, an initial gene-expression data set may include expression values for approximately 30,000 genes, and a digital pathology data set may include 41 metrics that characterize the spatial locations of cells. An integrative workflow may first identify a subset of genes and a subset of digital pathology metrics (or all digital pathology metrics) that are predicted to be sufficiently informative of progression, and an integrating model may then receive values for these subsets that pertain to a given subject to generate a result representing a prediction of progression. A combinatorial workflow may (for a given subject) predict: a first preliminary result representative of a prediction of progression based on expression of the 30,000 genes and a second preliminary result representative of predictive progression based on the 41 digital pathology metrics and may then generate a transformed result representative of predictive progression based on the first and second preliminary results. A hybrid workflow may first identify a subset of 30,000 genes for which expression levels are predicted to be informative of progression and to further generate (for a given subject) a preliminary result representative of predictive progression based on the 41 digital pathology metrics and may then generate a transformed result representative of predictive progression for the given subject based on the subject's expression levels for the subset of genes and based on the preliminary result.

Thus, one or more feature-focus machine-learning models can be used to shrink a variable set and/or to generate a predicted medical-condition state, a predicted medical-condition progression, or a predicted treatment response. Shrinking the variable set may allow for the integrating or hybrid model to be trained using a smaller training data set. Shrinking the variable set can also reduce a probability that the integrating or hybrid model will be trained with a data set that is too small, which can result in over fitting and bias.

Shrinking the variable set can include training a variable-focus machine-learning model using a bootstrapping technique to detect which variables were frequently selected as sufficiently contributing to a predicted output. The bootstrapping technique avails a different data set to the model during each of multiple iterations. This approach can be particularly advantageous when a training data set is relatively small: Whereas training a model using a data set with a high number of variables may result in over-fitting the data, bootstrapping aggregates results from iterations trained on different data sets, which can avoid over-fitting or a reduce an extent of over-fitting. Over-fitting can lead to bias in a model. Thus, both variable focusing and bootstrapping can reduce a probability that a model is biased and/or reduce an extent to which a model is biased. Given that an input data set fed to an integrating or hybrid machine-learning model is defined based on a shrunk variable set determined by one or more variable-focus models, using bootstrapping to train a variable-focus model can reduce bias in both the variable-focus model and the downstream (integrating or hybrid) model. These approaches can facilitate detecting various data patterns that pertain to individual subjects, time periods, or other circumstances while still capitalizing on higher level information that characterizes data across subjects, time periods, or other circumstances.

After the variables are selected using the bootstrapping technique, an integrating or hybrid machine-learning model can be trained to generate a result corresponding to a predicted medical-condition state, medical-condition progression, or treatment response based on the selected variables. During this training, parameters of the integrating or hybrid machine-learning model are learned, such that a specific value for each of multiple parameters is defined. This training may also use a Monte Carlo bootstrapping technique, where a different portion of a training data set is available to the model during each of multiple iterations. During each iteration, an interim set of parameter values are determined based on the portion of the training data set. The interim sets of parameter values are then collectively processed to determine a final set of parameter values for the integrating or hybrid machine-learning model.

After the integrating or hybrid machine-learning model is trained, the integrating or hybrid machine-learning model may receive an input data set that corresponds to a particular subject. The input data set can include expression levels of each of a set of genes and can include digital pathology data. The set of genes can selectively include genes identified using a variable-focus model. The digital pathology data can selectively include metrics identified using the same or different variable-focus models. The integrating or hybrid machine-learning model can generate a result that corresponds to a predicted state, progression, and/or treatment response based on the set of genes and the digital pathology data.

A diagnosis may be informed based on the predicted result. For example, the predicted result may correspond to a predicted disease, disease type, or disease stage. A care provider may use this result to inform a diagnosis. As noted above, some techniques disclosed herein include using a variable-focus model to perform variable focusing. For example, out of 30,000 genes, seven genes may be identified that are to be represented in a gene-expression input data. This variable reduction not only facilitates training of a downstream (integrating or hybrid) machine-learning model but also improves the interpretability. For example, in addition to outputting the predicted results, values fed to an integrating or hybrid model that generated the result may be output. Further, one or more “signature” or “fingerprint” value sets associated with a given disease, disease type, or disease stage can be output. Each of the signature value sets may include a representative value for each of the variables used by the integrating or hybrid model and/or a representative range of values for each of the variables (e.g., a mean value plus/minus two standard deviations). Thus, a care provider may be able to compare a subject's values to each of one or more signatures and use the comparison when determining whether he/she agrees with the result.

Alternatively or additionally, a treatment may be selected or recommended based on the predicted result. For example, a computing system may output a result that corresponds to a predicted probability of a medical condition progressing, and a medical provider may determine whether to recommend any treatment (versus monitoring the condition) based on the predicted probability. Other potential results include a predicted probability that the subject will survive for a predefined period of time, a predicted probability that the subject will survive without progression for a predefined period of time, a predicted probability that the subject's medical condition will advance to a particular stage (e.g., stage 4) within a predefined period of time, etc. A result may correspond to a prediction assuming that the subject does not receive treatment for the medical condition or a prediction assuming that a particular type of treatment is received by the subject. The assumed treatment or lack thereof can be based on a treatment that subjects represented in a training data set had received. For example, if the training data set included data corresponding to subjects that did not receive treatment (e.g., between a time at which input data was collected and when an output variable was observed), an output generated by a integrating, combinatorial, or hybrid machine-learning model trained using the training data may be interpreted as corresponding to a no-treatment assumption.

Further, one or more “signature” or “fingerprint” value sets associated with a given degree progression can be output. Each of the signature value sets may include a representative value for each of the variables used by one or more models generating a preliminary or final result (e.g., an integrating, hybrid, or combinatorial model or a model that generates a preliminary result that is fed to a combinatorial model) and/or a representative range of values for each of the variables (e.g., a mean value plus/minus two standard deviations). For example, a first signature may correspond to data values representing subjects for which no progression was observed, a second signature may correspond to data values representing subjects whose cancer progressed two stages within six months, etc. Thus, a care provider may be able to compare a subject's values to each of one or more signatures and use the comparison when determining whether he/she agrees with the result.

In some instances, models in a workflow (that may use the same model architectures) may be trained multiple times using a different data set that corresponds to a different type of treatment. For example, models in a data integration machine-learning workflow may be first trained using data from subjects with non-small-cell lung cancer treated with atezolizumab plus carboplatin plus paclitaxel (ACP) and may then be separately trained using data from subjects with non-small-cell lung cancer treated with bevacizumab plus carboplatin plus paclitaxel (BCP). Then, an input data set corresponding to a particular subject can be processed using each trained workflow to generate an output predicting a progression of a medical condition of the particular subject if the corresponding treatment is provided. A medical provider may then select a treatment associated with a lowest progression probability to recommend for the particular subject or may select a treatment by balancing progression predictions with adverse-event risks.

In some embodiments, a computer-implemented method can be provided, where the computer-implemented method can include a set of actions. The set of actions can include: accessing a first data set corresponding to one or more digital pathology images and to a particular subject; accessing a second data set corresponding to expression levels of a set of genes and to the particular subject; and generating a result that corresponds to a predicted current state of a medical condition or to a predicted progression of the medical condition, the result being generated by processing the first data set and the second data set using a machine-learning model.

In some instances, the set of actions includes one or more additional actions. An exemplary additional action includes: generating the second data set by filtering an initial set of expression levels of a larger set of genes, wherein the set of genes were identified using a variable-focus model configured to reduce an input data set. Another exemplary additional action includes: generating the first data set by processing the one or more digital pathology images using a first upstream machine-learning model, the first data set including a first preliminary result corresponding to a first preliminary prediction of the current state or to a first preliminary prediction of the medical condition. Yet another exemplary additional action includes: generating the second data set by processing the expression levels of the set of genes using a second upstream machine-learning model, the second data set including a second preliminary result corresponding to a second preliminary prediction of the current state or to a second preliminary prediction of the medical condition. Still other exemplary additional actions include: generating another result that corresponds to a another predicted progression of the medical condition assuming that the particular subject receives another particular treatment, the other result being generated by processing the first data set and the second data set using another machine-learning model; and selecting one of the particular treatment or other particular treatment to treat the particular subject or to recommend for treatment of the particular subject. Yet another additional action can include: selecting a treatment arm to which the particular subject is to be assigned based at least in part on the result and/or determining whether the particular subject is eligible to participate in a clinical study may be based at least in part on the result.

The first data set may include a set of spatial heterogeneity metrics identified by: detecting depictions of a set of immune cells in the one or more digital pathology images; detecting depictions of a set of tumor cells in the one or more digital pathology images; and generating each of the set of spatial heterogeneity metrics based on locations of the depictions of the set of immune cells and based on locations of the set of tumor cells.

The result may correspond to the predicted progression of the medical condition assuming that the particular subject receives a particular treatment. Additionally or alternatively, the result may correspond to the predicted progression of the medical condition and includes a probability of survival.

The medical condition may include a particular type of cancer.

In some embodiments, a computer-implemented method is provided that includes: accessing a data set corresponding to one or more digital pathology images and to a particular subject; and generating one or more predicted gene-expression levels by processing the data set using a machine-learning model.

The data set may include a set of spatial heterogeneity metrics identified by: detecting depictions of a set of immune cells in the one or more digital pathology images; detecting depictions of a set of tumor cells in the one or more digital pathology images; and generating each of the set of spatial heterogeneity metrics based on locations of the depictions of the set of immune cells and based on locations of the set of tumor cells.

In some embodiments, a computer-implemented method is provided that includes: accessing a data set corresponding to expression levels of a set of genes and to a particular subject; and generating one or more predicted digital pathology metrics by processing the data set using a machine-learning model.

In some embodiments, a computer-implemented method is provided that includes accessing a data set corresponding to one or more digital pathology images and to a particular subject; and generating one or more predicted gene-expression levels by processing the data set using a machine-learning model.

In some embodiments, a computer-implemented method is provided that includes accessing a data set corresponding to expression levels of a set of genes and to a particular subject; and generating one or more predicted digital pathology metrics by processing the data set using a machine-learning model.

In some embodiments, a method is provided that includes accessing a set of training data elements, each of the set of training data elements corresponding to an individual subject diagnosed with a medical condition. Each of the set of training data elements includes a first data set corresponding to one or more digital pathology images; a second data set corresponding to expression levels of a set of genes; and a label that indicates a state of the medical condition of the individual subject or a progression of the medical condition observed subsequent to a time point associated with collection of the one or more digital pathology images and to time point associated with collection of the expression levels. A machine-learning model is trained using the training data elements, where values for a set of parameters are learned during the training. A biomarker is determined for a particular type of state of the medical condition or a particular type of progression of the medical condition using at least one of the values for the set of parameters. An assay configured to detect the biomarker is provided.

In some embodiments, a computer-implemented method is provided that includes accessing a set of training data elements, each of the set of training data elements corresponding to an individual subject diagnosed with a medical condition. Each of the set of training data elements include: a first data set corresponding to one or more digital pathology images; a second data set corresponding to expression levels of a set of genes; and a label that indicates a state of the medical condition of the individual subject or a progression of the medical condition observed subsequent to a time point associated with collection of the one or more digital pathology images and to time point associated with collection of the expression levels. A machine-learning model is trained using the training data elements, where values for a set of parameters are learned during the training. A data signature for a particular type of state of the medical condition or a particular type of progression of the medical condition is determined using at least one of the values for the set of parameters, where the data signature includes a value or range for an expression level each of at least some of the set of genes and a value or range for each of one or more digital pathology metrics.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1 illustrates a process for training and using an integrated processing workflow to configure and train a model to predict a survivorship class based on gene-expression levels and/or spatial relationships between cells.

FIG. 2 shows the selection frequency for each of an exemplary set of genes to be in a preliminary reduced gene set that were determined to be predictive of a survivorship class.

FIGS. 3A and 3B show exemplary Overall Survival curves for subjects who received a baseline treatment and for a subpopulation of subjects who received another treatment, where the subpopulation was selected using a single-modality model.

FIG. 4 shows exemplary Overall Survival curves for subjects who received the baseline treatment and for a subpopulation of subjects who received another treatment, where the subpopulation was selected using a multi-modality approach.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

### I. Overview

There is high heterogeneity across subjects in data sets that may provide information about medical conditions (e.g., gene-expression and digital pathology data). Not only can there be high variability across subjects, medical-condition presentations, tumors, or tumor regions with respect to gene expression or digital pathology, but multiple distinct backdrops may give rise to the same gene-expression or digital pathology data set. For example, an expression level of a given gene may be influenced by whether a subject has another gene allele that inhibits, masks, or complements the gene. As another example, local oxygenation levels can affect cancer cell proliferation.

Further, these data sets typically have very high dimensionality. For example, gene-expression data can include expression levels for each of approximately 30,000 genes. A variable space can become even more complex if mutations are considered, as there are many tens of thousands of potential mutations that may occur in humans. As another example, digital pathology data can include multiple high-resolution images. The images may be pre-processed to identify portions of the images that depict cells of a given type (e.g., immune cells or tumor cells). Each cell depiction can then be represented by one or more locations (e.g., a center position or a boundary). A single slide may depict tens of thousands of cells of even a single type, and frequently multiple cell types are labeled using distinct stains.

The heterogeneity and large data sizes make it difficult to attempt to detect signals within the data that can be used to predict a current medical-condition state (e.g., disease, disease type, or disease stage) or future progression of a subject's medical condition (e.g., if a given treatment is received or without treatment administration). For example, generally, the amount of training data that is required to train a machine-learning model scales with a number of input features. With regard to some models (particularly deep neural networks), training data is frequently defined to include more individual labeled training elements than there are input features. However, securing this quantity of gene-expression and/or digital pathology data along with labels that characterize a current disease state or subsequent disease progression for a set of subjects having a same condition and receiving a same type of treatment (or lack thereof) can be incredibly costly and challenging and, in some cases, is not possible.

In some embodiments, a workflow is provided in which one or more machine-learning models process input data that includes multiple data types to generate a result corresponding to a predicted state or progression of a medical condition. The workflow may include an ordered set of actions that include one or more first actions that reduce the dimensionality of an input data set and one or more second actions that generate the result corresponding to the predicted state or progression based on the reduced-dimensionality input data set. The one or more first actions may include a pre-processing action. Input data that is processed by the workflow may include data of different types, and—in some instances—input data that is of each type is pre-processed (e.g., independently) and the pre-processed data is then collectively processed (e.g., using a data-integration machine-learning model). Actions involved in pre-processing data of a first type may be the same or different as actions involved in pre-processing data of a second type. Pre-processing may be performed using an upstream machine-learning model (e.g., corresponding to a particular type of data). For example, one upstream machine-learning model may be configured to transform, focus, or reduce one or more digital pathology images (or a set of digital pathology metrics) into a set of spatial heterogeneity metrics; another upstream machine-learning model may be configured to focus or reduce a number of gene-expression values in an input data set; and a data integration model may be configured to generate a result based on the set of spatial heterogeneity metrics and the focused gene-expression values.

Training models within an integrated processing workflow can include training one or more variable-focus models to adjust or improve a focus on variables or features that are more predictive of results than an initial set of variables or features. The focus adjustment may include reducing a dimensionality of a variable set or feature set. The focus adjustment may include identifying a subset of variables (i.e., to perform variable focusing) to use to predict the result. A set of parameters of a data integration machine-learning model can then be defined to support transforming values for the subset of variables into the predicted output. For example, a variable-focus machine-learning model can be trained to identify a subset of genes impacting disease progression or treatment response. A gene-expression input data set can then be shrunk to include expression values for the subset (and not for other genes). A data integration machine-learning model can be trained to transform input data set that includes the shrunk gene-expression input data set and data of another type (e.g., digital pathology data) into an output corresponding to the predicted state or progression. After training, expression data for the subset of genes can be accessed for a given subject and collectively processed with data of the other type (e.g., a set of spatial heterogeneity metrics associated with the given subject that characterize an extent to which immune cells are interspersed with tumor cells) to form a diverse data set (including data of multiple types). The collective processing can include using the data integration machine-learning model configured with the set of parameters learned during training to generate a result corresponding to a predicted state or progression. For example, the set of parameters may include a weight associated with each of one or more genes and/or a weight associated with one or more spatial heterogeneity metrics. As another example, the set of parameters may include an order number and/or threshold to be associated with a portion of a decision tree. As yet another example, the set of parameters may include a threshold to transform a gene-expression value or spatial hetereogeneity value into a binary number. The focusing of variables in this approach reduces the data being processed by the data integration machine-learning model, which can allow the data integration machine-learning model to be trained using a data set that is smaller than would be required to train a model that received the full variable set. Further, the integrative approach still allows a single model (the data integration model) to receive lower level data corresponding to each of multiple data types (e.g., to receive both gene-expression data and digital pathology data), such that the model may draw upon synergies in the data. For example, the data integration model may learn that a manner in which an expression of a given gene predicts a result can vary based on a particular digital-pathology metric.

Shrinking the variables can further improve the interpretability of the results generated by the data integration model and can facilitate determining signatures or watermarks for particular disease states or progressions. For example, a workflow may be configured to generate a predicted class that predicts a current stage of a particular type of cancer a subject has. To generate a signature for each disease stage, a training data set (e.g., that corresponds to the particular type of cancer) can be divided into subsets based on disease stages of the subjects. For each variable in the reduced variable set, a representative value or range for the variable can be determined for each of the disease stages based on the corresponding training-data subset. An output transmitted to a user device can include a result corresponding to a predicted stage for the subject, values of the reduced variable set for the subject, and can include one or more signatures. The user can then compare the subject-associated variable values with one or more signatures to understand how closely the subject's data matched values from each of multiple signatures.

While it can be advantageous to shrink a variable set, an integrative processing workflow need not involve variable focusing or shrinkage. Rather, the integrative processing workflow may include using a single machine-learning model (e.g., an data integration machine-learning model) to process data of different types to generate a result corresponding to a predicted medical-condition state or progression. For example, the data integration machine-learning model may process gene-expression data and digital pathology metrics. The gene-expression data may include expression values for all genes in the human genome or for a predefined subset of genes.

As another example, training models within a combinatorial processing workflow can include training each of multiple lower level models to learn a set of parameter values to support transforming an input data set corresponding to a particular data type (e.g., gene-expression data or digital pathology data) into a preliminary result corresponding to a medical-condition state or progression prediction. A higher level combinatorial machine-learning model can be trained to transform the preliminary results into a final predicted result corresponding to a predicted medical-condition state or progression. By initially separately processing data of different data types, the input data size stays limited and smaller than if the data were first aggregated and then processed using a single model. The constrained input data can allow the combinatorial machine-learning model(s) to be trained using a training data set that is smaller than would be required to train a model that received the full variable set. Further, the separate training may make it easier to collect a training data set for training a lower level model (that generates a preliminary result), because the training data set need not include each of the data types. For example, a first training data set may include gene-expression data and labels (identifying a medical-condition state or progression); a second training data set may include digital pathology data and labels; and a third training data set used to train the combinatorial (higher level) model may include gene-expression data, digital pathology data, and labels. The first training data set may be used to train a first lower level model, and the second training data set may be used to train a second lower level model. Parameters of the lower level models can then be initialized based on the training, and the third data set may be used to collectively train all three models.

As yet another example, training models with a hybrid processing workflow can include training a first technique to process a first set of variables corresponding to data of a first type (e.g., gene-expression data) and to identify a subset of the first set of variables and training a second technique to process a second set of variable values corresponding to data of a second type (e.g., digital pathology data) to generate a preliminary predicted result predicting a subject's medical-condition state or progression. A hybrid machine-learning model can be trained and configured to receive values for the first set of variables (e.g., values for a shrunk variable set) and the preliminary predicted result and to generate a transformed predicted result predicting the subject's medical-condition state or progression.

Using a workflow that processes multiple types of data may be particularly advantageous when input data is noisy and/or high-dimensional. For example, gene-expression data may be generated by collecting and processing a sample. However, the sample may include multiple micro-environments, and the expression of various genes may vary across the micro-environments. Collectively analyzing another type of data (e.g., digital pathology data) with the gene-expression data may provide information as to how the expression levels may be interpreted when considering a particular slice.

Post-hoc analysis may even be able to predict whether particular features in digital pathology data are predictive of expression levels of particular genes. Potentially, collection of gene-expression data may be avoided if a digital-pathology feature is detected from which particularly relevant gene-expression data can be inferred. Conversely, collection of a sample for digital pathology may be avoided if expression of one or more genes predict particularly pertinent digital pathology features with sufficient confidence.

### II. Exemplary Types of Input Data

A workflow (e.g., an integrative processing workflow, a combinatorial workflow, or a hybrid workflow) may be configured to process data corresponding to multiple data types. Further, a single machine-learning model (e.g., a data integration machine-learning model, a combinatorial machine-learning model, or a hybrid machine-learning model) can be configured to process data corresponding to multiple data types.

The multiple data types may correspond to data collected using different types of techniques. The multiple data types may include (for example) two or more of gene-expression data, digital pathology data, gene-mutation data, and radiology data.

II.A. Gene-Expression Data

Gene expression may be measured using (for example) RNA-Sequencing (RNA-Seq), serial analysis of gene expression (SAGE), rapid analysis of gene expression (RAGE), Northern blotting, Southern blotting, real-time PCR, a gene chip, or a microarray. An expression level of a gene can be estimated based on a quantity of RNA. For example, RNA-Seq uses next-generation sequencing to collect a set of reads, which are then assembled or aligned to a reference genome. Gene expression is then estimated based on a number of reads mapped to each locus and one or more normalization factors (e.g., sequencing depth, gene length, or total sample RNA output). Thus, gene-expression data can include an expression level for each of one or more genes in the genome.

Gene-expression data can include a value for each of one or more gene-expression variables, where each gene-expression variable corresponds to a particular gene. The value may be (for example) numeric or categorical. A categorical value may indicate (for example) whether expression of the gene is zero, low, average, or high as compared to a population. The population may include a healthy population or a population of subjects with a particular medical condition.

II.B. Digital Pathology Data

Digital pathology data can be obtained by collecting a sample (e.g., biopsy) from a subject. The sample can be fixed and sliced. One or more stains can be applied to each slice, and the slice can be imaged. Each stain may be selected based on a selective absorption by a cell, organelle, or structure of interest. For example, Ki-67 may be selectively absorbed by tumor cells. In some instances, a single stain is absorbed by multiple types of cells (e.g., both immune cells and tumor cells) and an image analysis predicts whether each individual cell is a tumor versus an immune cell based on the cell's morphology. In some instances, a stain may be used that is absorbed by surface receptors of a given type, to facilitate detecting even different types of immune cells.

Each image can then be processed to infer a location of each cell of a particular type (e.g., each immune cell and tumor cell). The location may be represented by (for example) a point location or boundary. In some instances, digital pathology data that is processed using a workflow disclosed herein includes the image itself and/or a representation of cells of a particular type.

In some instances, digital pathology data that is processed using a workflow disclosed herein includes a digital pathology image. The workflow may include processing the digital pathology image using an image-processing technique and/or neural network (e.g., a convolutional neural and/or deep neural network). An output generated by the image-processing technique and/or neural network may include (for example) a predicted state or predicted progression of a subject, one or more spatial heterogeneity metrics, etc.

In some instances, digital pathology data that is processed using a workflow disclosed herein includes one or more metrics that represents an absolute or relative quantity and/or an absolute or relative location of cells (or other biological structures) of a particular type. For example, a workflow can include detecting each depicted immune cell and tumor cell in an image and identifying a point location for each cell. The workflow can further include determining a spatial heterogeneity metric that characterizes a degree to which immune cells are interspersed with a set of tumor cells. The metric may be determined (for example) based on the cells' point locations or based on a density or count of one or more cell types within individual regions or an image. The metric may be determined by using (for example) a spatial-point-process analysis framework, a spatial-areal analysis framework, or a geostatistical analysis framework. Exemplary techniques for determining digital pathology data (e.g., spatial heterogeneity metrics) are disclosed in U.S. Provisional Application No. 63/026,545, titled “Predicting Treatment Response of Non-Small Cell Lung Cancer Subjects” and filed on May 18, 2020 and in U.S. Provisional Application No. 63/077,232, titled “Spatial Feature Analysis for Pathology Slide Images” and filed on Sep. 11, 2020. Each of these applications is hereby incorporated by reference in its entirety for all purposes.

Digital pathology data associated with a particular subject and/or a particular time point and that is used to generate a particular result (predicting a medical-condition state or progression) may include a digital pathology data set that includes at least 1, at least 2, at least 3, at least 5, at least 10, at least 15, at least 20, at least 25, at least 30, at least 35, at least 40, at least 45, or at least different types of digital pathology variables (e.g., spatial heterogeneity metrics). Digital pathology data associated with a particular subject and/or a particular time point and that is used to generate a particular result (predicting a medical-condition state or progression) may include a digital pathology data set that includes fewer than 500, fewer than 200, fewer than 150, fewer than 100, fewer than 90, fewer than 80, fewer than 70, fewer than 60, or fewer than 50 different types of digital pathology variables. The digital pathology data set may include at least 1, at least 2, at least 3, at least 4, at least 5, at least 8, or at least 10 different types of spatial heterogeneity metrics generated by processing digital pathology images as point pattern data; at least 1, at least 2, at least 3, at least 4, at least 5, at least 8, or at least 10 different types of spatial heterogeneity metrics generated by processing digital pathology images as areal data; and/or at least 1, at least 2, at least 3, at least 4, at least 5, at least 8, or at least 10 different types of spatial heterogeneity metrics generated by processing digital pathology images as geostatistical data.

Digital pathology data can include a value for each of one or more digital pathology variables. The value may be (for example) numeric or categorical. A categorical value may indicate (for example) whether the value is low, average, or high as compared to a population. The population may include a healthy population or a population of subjects with a particular medical condition.

II.C. Gene-Mutation Data

Genetic mutation data can identify particular variants in a subject's DNA. A variant can include a single nucleotide polymorphism or a structural variant (e.g., a copy number variant, deletion, insertion, translocation, or inversion). Genetic mutation data may further include a tumor mutational burden, chromotrypsis events, and/or mutation predictors (smoking history or extensive ultraviolet light exposure).

A variant may be identified by using direct sequencing, collecting a set of reads, aligning each read to a reference sequence, determining a sequence for the subject based on the aligned reads, and comparing the subject's sequence to the reference sequence to detect variants. Alternative techniques that can be used to detect a variant include DNA hybridization, restriction enzyme digestion, or using a DNA chip.

Gene-mutation data can include a value for each of one or more variant variables, where each variant variable corresponds to a particular potential variant. The value may be (for example) binary and indicate whether the variant was detected for a particular subject. In some instances, the value is numeric and may represent a copy number. The value may be numeric and represent an estimated probability that the subject has the variant.

Gene-mutation data may be identified by processing a sample, such as a biopsy of a tumor or a liquid biopsy. For example, genetic mutation data may identify one or more mutations in circulating tumor DNA (ctDNA).

II.D. Radiology Data

Radiology data can characterize (for example) a PET scan, CT scan, x-ray or MRI data associated with a subject. The radiology data can include one or more images (e.g., CT images, x-ray images, MRI images, PET-scan images), which may be subsequently processed by a machine-learning model (e.g., a convolutional neural network and/or a deep neural network) to predict one or more tumor metrics. The radiology data may include the one or more tumor metrics.

A tumor metric may identify (for example) a shape, perimeter, aspect ratio, area, or volume of one or more tumors. The radiology data can identify a cumulative area of cumulative volume of multiple tumors. The tumor metric may identify a number of organs in which there were observed to be one or more tumors.

### III. Exemplary Machine-Learning Models

A workflow (e.g., an integrative processing workflow or a hybrid processing workflow) can include using one or more variable-focus machine-learning models to identify a reduced variable set and/or a disease-prediction model (e.g., a data integration model, hybrid model, combinatorial model, or lower level model feeding to a combinatorial model) to generate a result corresponding to a predicted disease state or progression.

III.A. Variable-Focus Models

The reduced variable set can be identified by the variable-focus machine-learning model by predicting which variables are most predictive of a result variable (e.g., characterizing a medical-condition state or disease progression). The variable-focus model may receive one or more types of data identified in section II and may reduce a variable count, reduce a variable dimensionality, and/or transform a variable space so as to transform the data. For example, a variable-focus model may be configured to identify an incomplete subset of a set of genes for which expression values are or may be available, where the incomplete subset represents genes for which expression values are more representative of a result (e.g., a current state or predicted progression of a medical condition) relative to genes not in the subset. As another example, a variable-focus model may be configured to identify an incomplete subset of a set of spatial heterogeneity metrics that are or may be available, where the incomplete subset represents metrics for which values are more representative of a result (e.g., a current state or predicted progression of a medical condition) relative to values not in the subset.

The one or more variable-focus machine-learning models may include (for example) a regression model, such as a linear regression model, a logistic regression model, a Ridge regression model, or a least absolute shrinkage and selection operator (Lasso) model. For example, the regression model may assign a weight to each of an initial variable set (e.g., each of a set of genes in a gene-expression data set), and a reduced variable set may be defined to include those variables assigned a weight above a given threshold or a particular number of variables assigned the highest weights.

The one or more variable-focus machine-learning models may be used to aggregate multiple variables. For example, a matrix-decomposition or clustering technique may be used to identify one or more particular components (e.g., a first component) or one or more particular clusters (e.g., a largest cluster). Each component may be defined to be a weighted average of variables, and the weighting may then be used to collectively assess multiple variables downstream. Similarly, each cluster may be defined based on statistics of the cluster (e.g., a mean and standard deviation for each of multiple variables), and the statistics may be used to subsequently aggregate values from the multiple variables.

The one or more variable-focus machine-learning models may include (for example) a clustering model, such as a component-analysis clustering model (e.g., that uses Principal Component Analysis or Independent Component Analysis), or a hierarchical clustering model. A reduced variable set may be defined to include variables that are highly represented in (for example) one or more prominent components (e.g., a first component, second component, and/or third component) or that are associated with higher levels in a clustering hierarchy.

The one or more variable-focus machine-learning models may include a tree-based approach (e.g., that uses one or more decision trees or a random survival forest). A reduced variable set may be defined to include variables that are included in at least a threshold number (e.g., at least one) of decision nodes of that are assigned at least a threshold weighting value.

The one or more variable-focus machine-learning models can use BORUTA feature selection, which can identify features that convey information about a result that exceeds information predicted by permuted features. More specifically, for each of multiple evaluation runs, a “shadow copy” is created for each feature, where the shadow copy corresponds to a permuted version of the feature. A random forest model is trained at each evaluation, which identifies a contribution of each feature and of its shadow copy to a result. Features that are informative as to a result are identified as those for which the contribution of the feature was better than that of its shadow copy at least a threshold percentage of times (e.g., at least 90%). If features show very strong performance across initial evaluation runs (e.g., where the contribution of the feature exceeds that of its shadow copy for 9 or 10 times out of 10 initial evaluation runs), the strong features are removed from the training set to give “weaker” signals a chance of being selected by the random forest.

The one or more variable-focus machine-learning models may include a neural network (e.g., a feedforward neural network and/or convolutional neural network). A reduced variable set may be defined to include variables determined as having a relative importance of each variable in predicting an output that is above a predefined absolute or relative threshold. A relative importance may be determined (for example) by using a technique disclosed in arson, G. D. 1991. Interpreting neural network connection weights. Artificial Intelligence Expert. 6(4):46-51 or by using a randomization approach, such as a technique disclosed in Olden, J. D., Jackson, D. A. 2002. Illuminating the ‘black-box’: a randomization approach for understanding variable contributions in artificial neural networks. Ecological Modelling. 154:135-150. Each of these references is incorporated by reference in its entirety for all purposes.

For example, an integrative processing workflow may use a variable-focus model that uses a component analysis (e.g., principal component analysis) to transform (e.g., reduce) a dimensionality of an input data set, and a data integration model that uses clustering technique (e.g., k-means clustering) may then be used to generate a result based on the reduced dimensionality input data set.

As described herein, a variable-focus machine-learning model shrinks an input data from an initial size to a smaller size. In some instances, the smaller size is predefined. For a given type of data, the smaller size may be defined to have a specific quantity of variables, where the quantity of variables is defined to be less than 500, less than 250, less than 100, less than 50, less than 25, less than 15, less than 10, less than 8, or less than 6 variables. Alternatively, the smaller size may be defined to be a fraction of an initial size of the input data (potentially with a lower bound and/or upper bound). For example, the smaller size may be defined to less than 80%, less than 50%, less than 30%, less than 20%, less than 15%, less than 15%, less than 10%, or less than 5% of the initial size. Alternatively, the reduced variable set to selectively include each variable for which a condition was satisfied (e.g., a contribution weight or relative importance was above a predefined threshold. As exemplary illustrations: a reduced variable set may be defined to include precisely 8 variables; a reduced variable set may be defined to include 5% of the variables represented in an initial variable set (e.g., rounding up, if needed); or a reduced variable set may be defined to include each variable that was associated with a weight in any of the first through third components that is above 0.02).

III.B. Disease-Prediction Models

A disease-prediction model can include a model that generates a result corresponding to a predicted medical-condition state (e.g., a predicted current disease state) or a predicted progression. A predicted medical-condition state may predict (for example) a particular stage of cancer, a particular type of disease, a particular severity of disease, a particular type of immune activity, etc. The prediction may include a predicted progression assuming that the subject receives a particular type of treatment. A predicted progression of a medical condition may predict whether a subject achieves remission within a particular time period, whether no progression of the subject's medical condition is observed across a particular time period, whether a particular type or level of progression of the subject's medical condition is observed within a particular time period, whether at least a particular type of level of the subject's medical condition is observed across a particular time period, whether the subject survives for at least a particular duration, and/or for how long the subject survives. A predicted progression of a medical condition may predict whether the subject A predicted progression of a medical condition may include predicting that the subject will be part of a longer survival class (or a shorter survival class). The shorter survival class may include subjects that survived for less than a threshold period of time from treatment initiation.

Each of a data integration model, hybrid model, or combinatorial model is a disease-prediction model. A lower level model that feeds a preliminary result to a hybrid or combinatorial model can also be a disease-prediction model.

A disease-prediction model can include (for example) any of the types of models identified in Section III.A. If a workflow uses multiple models (e.g., a variable-focus model and a disease-prediction model), two or more of the multiple models may have a same architecture and/or two or more of the multiple models may have a different architecture. If a workflow includes use of a variable-focus model and a disease-prediction model, a quantity of variables represented in an input for the variable-focus model may be larger than a quantity of variables represented in an input for the disease-prediction model.

For example, each of the variable-focus model and the disease-prediction model may include a Lasso model. The variable-focus model may be configured to receive values for approximately 30,000 gene-expression variables (e.g., corresponding to different genes). The variable-focus model may then identify a subset of the gene-expression variables (e.g., corresponding to 7 genes). The disease-prediction model may be configured to receive values for the subset of gene-expression variables and values for a set of digital-pathology variables (e.g., corresponding to 41 digital-pathology metrics) and generate a result.

In some instances, a disease-prediction model includes an ensemble model. For example, parameters for multiple variable-focus models may be defined based on different data cuts. Each of the multiple variable-focus models may feed a result to a low-level disease-prediction model which may generate a low-level result (based on the fed result and potentially other data), and the ensemble model may then determine a final result based on multiple low-level results.

In some instances, preliminary parameter values for a disease-prediction model are defined based on results from different variable-focus models. The preliminary values of the parameters of the disease-prediction models may be collectively analyzed to define final parameter values for the disease-prediction model.

As further explained in Section V.A. below, after one or more models in a processing workflow are trained, input data corresponding to a different subject (e.g., not represented in a training data set) can be received. The input data can include one or more data types identified in Section II. Part or all of the input data may be pre-processed (e.g., to detect locations of cell depictions, identify a cell type of each cell depiction, perform a normalization, perform a standardization, generate a spatial heterogeneity metric, and/or filter a variable set to generate a focused variable set). In some instances, part of the input data (which may include part of the original input data and/or a pre-processed version of part the original input data) is then fed to a trained upstream model to generate a preliminary result that corresponds to a predicted medical-condition state or a predicted progression of a medical condition. One or more preliminary results can then be fed to a result-prediction model (e.g. a combinatorial model or hybrid model) to generate a result that corresponds to a predicted medical-condition state or predicted progression of a medical condition. In some (same or different) instances, part or all of the input data (which may include part or all of the original input data and/or a pre-processed version of part or all the original input data) is fed to a trained result-prediction model (e.g., a data integration model or hybrid model) to generate a result that corresponds to a predicted medical-condition state or predicted progression of a medical condition.

As further described in Section V.B. below, a trained result-prediction model may further be used to identify a signature value set representative of a particular type of subject sub-population.

As further described in Section V.C. below, a trained result-prediction model, a variable-focus model, or an upstream model that feeds to a trained result-prediction model may be used to select one or more biomarkers (e.g., predictive of a current medical-condition state or of a progression of a medical condition) and/or to develop an assay.

As further described in Section V.D. below, a result that corresponds to a predicted medical-condition state or predicted progression of a medical condition may be used to inform a selection of a treatment for the subject, a determination as to whether the subject is eligible for a clinical study, an assignment of the subject to a particular arm in a clinical study.

As further described in Section V.E. below, a trained result-prediction model may be used to infer one or more cross-modality dependencies.

### IV. Exemplary Training

A machine-learning model described herein (e.g., a variable-focus model, disease-prediction model, or a model described in section III) and/or a machine-learning model configured to process data of a type described herein (e.g., input data described in section II) can be trained in an iterative and/or sequential manner. The training may include using cross-validation and/or bootstrapping. The training may include using a technique described in one or more of sections IV.A, IV.B and/or IV.C.

IV.A. Training Workflows with a Variable-Focus Model

In an integrative processing workflow and a hybrid processing workflow (that includes a model described in section III and/or configured to process a type of data identified in section II), a variable-focus machine-learning model selects at least some of the variables that are to be included in data sets input to a data integration or hybrid model. One training approach is to train the variable-focus machine-learning model (such that variables to be input to the data integration or hybrid model are selected) and to then train the data integration or hybrid model. This may be done multiple times and/or iteratively.

For example, during each of multiple independent training iterations, the variable-focus machine-learning model can identify a subset of variables (e.g., a subset of input variables representing gene-expression data, digital pathology data, gene-mutation data, or radiology data as identified in any of sections II.A-II.D) to be included in an input to a data integration or hybrid model, and the data integration or hybrid model can be trained to assign a weight to each of the subset of variables. The subset of variables may include (for example) expression levels that correspond to a subset of genes and/or a subset of spatial heterogeneity metrics representing relative locations of immune and tumor cells in digital pathology images. Subsequent to these iterations, the weights may be collectively assessed to determine which variables were assigned the highest weights. To illustrate, consider a circumstance where the variable-focus machine-learning model identified variables A, B, and C to be in a variable subset in a first iteration and variables A, B, and D to be in a variable subset in a second iteration. If a downstream model assigns weights 0.6, 0.35, and 0.05 to variables A, B, and C (respectively) in the first iteration and weights 0.4, 0.2 and 0.2 to variables A, B, and D (respectively) in the second iteration, a cross-iteration assessment may determine that variables A, B, and D are to be included in the variable subset.

A cross-iteration assessment may be performed to (for example) define a input set to include each variable associated with a weight during any iteration above a predefined threshold, each variable associated with a mean or median weight across iterations above a predefined threshold, a predefined quantity of variables selected as those associated with a highest maximum weight assigned in an iteration (e.g., 15 variables associated with highest weights), a predefined quantity of variables selected as those associated with a highest median or average weight assigned across iterations. In some instances, variable selection may further depend on a number or fraction of iterations where a variable-focus machine-learning model selected a variable to be in a variable subset. For example, a cross-iteration assessment may associate each variable not included in the variable subset with a weight of zero during those iterations.

As another example, during iterative training, a quantity of variables that are included in a variable subset may gradually decrease over iterations, and weights assigned by a downstream model (e.g., a data integration or hybrid machine-learning model) may be used in a subsequent selection of a variable subset. For example, during a first iteration, a variable-focus model may identify 100 variables to be included in a variable subset, and a downstream model may then assign a weight to each of the 100 variables. During a next iteration, a variable-focus model may identify a preliminary score (e.g., representing a contribution weight or variable selection) for each variable in an initial set, and each preliminary score can be adjusted (e.g., where preliminary scores for variables associated with high weights in a previous iteration are boosted and/or where preliminary scores for variables associated with low weights in a previous iteration are reduced). A smaller variable set (e.g., of 90 variables) can then be selected based on the adjusted scores. Iterations may continue in this manner until a target number of variables are selected or a predefined number of iterations are completed.

IV.B. Training Workflows with a Model that Predicts a Preliminary Result

In a combinatorial processing workflow and a hybrid processing workflow (that includes a model described in section III and/or configured to process a type of data identified in section II), an upstream machine-learning model generates a preliminary result based on data of a given type, and the result is fed to a downstream model to process the preliminary result in with one or more other data points associated with another data type.

In some instances, the upstream machine-learning model that generates a preliminary result is trained separately from the downstream machine-learning model. For example, a preliminary result may correspond to a predicted current stage of a medical condition, and a loss function can be configured to introduce a penalty that scales with a degree to which the predicted stage differs from an actual stage. A loss function that is used to train the upstream machine-learning model may be the same as or different from a loss function used to train the downstream model.

In some instances, the upstream machine-learning model that generates a preliminary result is trained with the downstream machine-learning model. In this instance, an accuracy of the preliminary result need not be separately evaluated. Rather, a loss function evaluates an accuracy of a result generated by the downstream machine-learning model, and feedback can be provided to both the upstream and downstream machine-learning models.

In some instances, the upstream machine-learning model that generates a preliminary result is initially trained separately, where a loss function is used to introduce penalties based on accuracies of preliminary results. Parameters of the upstream machine-learning model can then be initialized with values learned during the initial training, and the upstream and downstream models can then be trained together.

IV.C. Bootstrapping and Cross-Validation Methodology

Bootstrapping includes randomly resampling a data set with replacement. ddCross-validation includes splitting a data set into multiple portions (e.g., with each portion being used to either train, validate or teat a model). Bootstrapping and/or cross-validation may be used to identify a subset of variables by a variable focus model. For example, bootstrapping may be used for stability selection of genes for which expression levels are most predictive of a result. This bootstrapping may be performed in a nested loop with Monte Carlo cross-validation. Thus, the Monte Carlo cross-validation can result in repeatedly redefining which data is assigned to training, validation, and testing portions, and the bootstrapping technique can be used to repeatedly resample each of one or more portions to generate interim results.

Bootstrapping and/or cross-validation may be used to identify a subset of variables to be input into a given model (e.g., via a variable-focus model, such as one described in section III.A) and/or to determine how to transform an input data set (e.g., that may include raw data, pre-processed data, filtered data, and/or a preliminary result) into a result (e.g., via a disease prediction model, such as one described in section III.B). For example, bootstrapping and/or cross-validation may be used to identify a subset of genes to be provided as input to a disease prediction model, to identify a subset of spatial heterogeneity metrics to be provided as input to a disease prediction model, and/or to identify one or more weights to be assigned to input variables received by a disease prediction model to generate a result. Each resampling and/or re-partitioning of the data may identify different variable selections and/or weights, which may subsequently be collectively processed to identify a final variable selection and/or weight.

Bootstrapping and/or cross-validation can be particularly advantageous when a number of model parameters is high, when a training data set is small, and/or when a training data set (and/or data set on which the model is to be used) has high variance. Further, iterations used in bootstrapping can be performed in parallel, which can speed a training time.

In some instances, bootstrapping and/or cross-validation can be used to train one or more upstream models (e.g., a variable-focus machine-learning model), to train one or more downstream models (e.g., a data integration machine-learning model, a combinatorial machine-learning model, or a hybrid machine-learning model), and/or to collectively train one or more upstream models with one or more downstream models.

For example, a narrow data set that includes labels corresponding to a specific type may be processed to produce multiple first subsets, each of which are used to identify a particular variable subset. An upstream machine-learning model may then generate a predicted result using the variable subset. This process may be repeated (each time changing the subset produced from the narrow data set). These predicted results can be aggregated and used to provide feedback to the downstream and/or upstream models.

As another example, a training data set may be divided into half (or other percentage division). A first half may be used to train one or more upstream models using bootstrap and/or cross-validation training. The upstream model(s) can be initialized with the values learned during this training, and the upstream and downstream models can then be collectively trained using the second half of the training data set. This collective training may, but need not, use bootstrap and/or cross-validation training.

Bootstrapping can support stability selection, where one or more variables are defined based on processing of multiple different (overlapping or non-overlapping) data sets. Stability selection can be particularly advantageous when input data exhibits high heterogeneity across subjects and/or time.

### V. Exemplary Use of Trained Model(s)

Once models in a processing workflow are trained, at least one of the models can be used to generate a result (e.g., that corresponds to a predicted state of a medical condition or a predicted progression). It will be appreciated that, in some instances, one or more of the models included in a processing workflow are not used after training. For example, a variable-focus machine-learning model may be used to select a reduced variable set during training but need not be used after training.

V.A. Predicting a Current State or Future Progression of a Medical Condition for a Specific Subject

After one or more models in a processing workflow are trained, input data corresponding to a different subject (e.g., not represented in a training data set) can be received. The input data may be pre-processed to transform at least part of the input data. For example, the pre-processing may include normalizing a value; normalizing or cropping an image; detecting a location of each depicted cell of one or more types; and/or generating one or more metrics based on an image (e.g., generating one or more spatial heterogeneity metrics based on detected cell locations). The pre-processing may include selecting a subset of variables associated with a given data type that correspond to a selected reduced variable set (e.g., which may be included in an input data set with one or more variables associated with another data type). As an illustration, a digital pathology image may be cropped and then processed to detect a location of cells of each of two cell types; a set of spatial heterogeneity metrics can be determined based on the cells' locations; and the set of spatial heterogeneity metrics can be included in an input data set with a subset of available gene-expression values, where the subset corresponds to a defined set of genes.

In some instances, at least part of the pre-processed data is fed to an upstream machine-learning model to generate a preliminary result. In some (alternative or same) instances, at least part of the pre-processed data is fed to a downstream machine-learning model (which may also receive a preliminary result).

A model that receives the pre-processed (or original) input data can be configured with parameter values learned during training. The model may include (for example) a data integration model, a hybrid model, a combinatorial model, or a model upstream of a hybrid or combinatorial model. A data integration, combinatorial, or hybrid model may generate a result that corresponds to a predicted medical-condition state or a predicted progression of a medical condition. An upstream model may generate preliminary results that correspond to a predicted medical-condition state or a probability of progression of a medical condition, and the preliminary result can be fed to another model (e.g., that may also receive other data that includes or is based on data of another type) to generate a result.

It will be appreciated that, after training, a workflow (e.g., an integrated processing workflow or hybrid processing workflow) may be reduced to omit one or more variable-focus models. Rather, variable focusing may be automatically performed in accordance with a reduced variable set identified as a result of training.

A result that corresponds to a predicted medical-condition state or a predicted progression of a medical condition can be used to determine or recommend whether to prescribe a particular medical treatment (e.g., associated with training data used to train the model) to the subject. A predicted progression of a medical condition may include predicting that the subject will be part of a longer survival class (or a shorter survival class). The shorter survival class may include subjects that survived for less than a threshold period of time from treatment initiation.

V.B. Signature Generation

A trained result-prediction model, a trained variable-focus model, a trained upstream model, training data and/or subsequent data can be used to generate a signature value set representative of a particular type of subject sub-population of a population. The population can correspond to subjects who have been diagnosed with a particular medical condition. The population may correspond to subjects with one or more additional or alternative constraints, such as having a particular genetic mutation, corresponding to a particular demographic profile, having a particular treatment history, etc.

The sub-population may correspond to a subset of subjects within the population. The sub-population may correspond to subjects that are determined (e.g., subsequent to a time at which input data was collected) to have a particular disease state (e.g., a particular stage of cancer, a particular type of disease, a particular severity of disease, a particular type of immune activity, etc.) or to exhibit a particular subsequent type of progression (e.g., achieving remission within a particular time period, no progression across a particular time period, a particular type or level of medical-condition progression within a particular time period, at least a particular type level of medical-condition progression within a particular time period, surviving for at least a particular time period, etc.). The sub-population may correspond to subjects that achieved an immunological response within a predefined range at a time corresponding to a collection of input data or at a predefined subsequent time. The immunological response may be estimated based on (for example) a spatial heterogeneity metric.

The signature value set can include (for example) a representative value or range for each of one or more variables input to one or more models in a processing workflow (e.g., a data integration model, hybrid model, combinatorial model or an upstream model that feeds to a hybrid or combinatorial model). For example, the signature value set can include a representative value or value range for each variable in a reduced variable set that is fed to a data integration model or hybrid model. The signature value set can also or alternatively include a representative value or value range for each variable fed to an upstream model that generates a preliminary result that is fed to a hybrid model or a combinatorial model.

Representative values or value ranges may be determined using (for example) statistics, a Monte Carlo technique, a distribution analysis, and/or a multi-variate analysis. For example, a data set (e.g., a training set or retrospective data set) may be filtered to select data elements corresponding to subjects associated with labels or retrospective data points indicating association with a sub-population. The selected data elements may then be assessed to identify (for example) a mean, median, mode, range (e.g., total range or range defined based on a mean plus/minus a particular number of standard deviations) for each variable. In some instances, a statistic or range can be identified for each variable independently. In some instances, statistics or ranges are determined based on a multi-variate distribution across the select data elements.

Representative values or value ranges may be determined using bootstrapping. For example, different slices of data may be analyzed in each iteration, and specific data elements corresponding to a particular sub-population can be identified for each iteration. The specific data elements can be analyzed to determine (for example) representative values or statistics for data variables. The representative values may be determined independently across variables or may be determined in a manner that considers variable dependencies. For example, if representative values are to be determined, a representative value may be independently stochastically identified for each variable or a representative data set (e.g., corresponding to a single subject and/or data collection) may be identified.

Signature value sets may be used to generate a result for given input data. For example, a machine-learning model may determine to which of multiple signature value sets a subject's input data most closely corresponds. The determination may be based on (for example) a clustering technique, distance-based technique or nearest-neighbor technique. A predicted medical-condition state or progression may then be identified as one that corresponds to the signature value set to which it was determined that the subject's input data most closely corresponds.

An output availed to a user may identify the signature value set determined to most closely correspond to a subject's data and/or a predicted medical-condition state or progression associated with the signature value set. In some instances, the output further includes one or more other signature value sets, which may convey an extent to which the subject's data more closely corresponded with the signature value set relative to other signature value sets.

V.C. Biomarker Identification/Assay Development

An integrated processing workflow or a hybrid processing workflow can include a variable-focus model that can be trained to identify a reduced variable set. In some instances, an upstream model may be trained to learn weights corresponding to individual variables in the reduced variable set. Thus, variables in the reduced variable set may be interpreted as being predictive of a result of interest and any downstream weighting may further inform the degree to which individual variables in the reduced variable set are predictive of the result. A machine-learning model (e.g., an upstream machine-learning model feeding to a combinatorial or hybrid machine-learning model) may further include parameters that indicate an extent to which an individual variable is predictive of a result, and/or a machine-learning model may be probed to estimate an extent to which one or more individual variables are predictive of a result. Thus, one or more variables may be identified as being predictive of a result, and/or an extent to which each of one or more variables are predictive of a result can be estimated.

At least one variable may then be selected to use in a biomarker assessment. For example, it may be determined that the expression levels of at least one gene and/or the values of at least one digital pathology metric are biomarkers for a current medical-condition state or future medical-condition progression. The at least one variable may be determined based on (for example) a predefined threshold for a weight and/or significance.

An assay may then be developed to determine whether and/or an extent to which the biomarker(s) are present for a given subject. For example, the assay may be configured to assess expression levels of the at least one gene and/or to assess values for the at least one digital pathology metric.

V.D. Clinical Study Design

A model described herein (e.g., a data integration model, hybrid model, or combinatorial model) can be used to generate a result that predicts a current medical-condition stage or future progression. This result may assume the existence of a particular diagnosis, treatment history, demographic constraints, and/or a subsequent treatment. For example, a result may predict whether a subject diagnosed with non-small-cell lung cancer will survive at least 24 months if a particular treatment (e.g., a standard of care treatment) is initiated at a baseline time.

This type of prediction may be used to constrain who is eligible to participate in a clinical study and/or how a cohort is defined for the study. For example, study criteria may indicate that, in order to be eligible, a result generated by a model must predict that the probability that the subject will survive for at least 24 months falls within a predefined range. As another example, subjects may be divided across multiple arms in a study in a manner such that the survival probabilities as calculated using the model are not significantly different.

V.E. Cross-Modality Data Inference

Training a machine-learning model can result in values of a set of parameters being learned. In some instances, at least one parameter value indicates a dependency or relationship between two variables. In some instances, the trained model can be probed to identify a dependency or relationship between two variables. In some instances, training of the model can be controlled to identify a dependency or relationship between two variables.

For example, a machine-learning model (e.g., a data integration machine-learning model) may first be trained using input data corresponding to two data types (e.g., gene-expression levels and digital pathology metrics). Learned parameter values may include a weight associated with each of multiple genes and each of multiple digital pathology metrics. The model may then be trained again with training data that omits one, more or all digital pathology metrics. Weights associated with each gene-expression variable may be compared across the training instances, and strategic training iterations may uncover how a weight associated with a given gene depends on the presence or absence of one or more digital pathology metrics. It will be appreciated that a similar technique may be applied by comparing which genes were represented in a reduced variable set in various training instances and how such representation depended on which digital pathology metrics were included in the training data.

As another example, training a machine-learning model may include defining a covariance matrix, which may indicate dependencies between various variables.

Relationships between variables may allow for a particular type of data collection to be avoided in at least some instances. For example, it may be determined that immune cells and tumor cells being interspersed in digital pathology images is indicative of high expression of one or more genes. Thus, if it is observed that the immune cells and tumor cells are interspersed in digital pathology images for a subject (or if a corresponding spatial heterogeneity metric indicates such interspersing), a care provider may refrain from requesting genetic testing.

### VI. Example

This Example relates to determining the extent to which an integrated processing workflow can be used to identify characteristics of a subpopulation that exhibit a particular type of treatment response.

VI.A. Background/Objective

More specifically, the IMpower150 study was a phase 3 clinical trial that evaluated the efficacy of cancer immunotherapy bevacizumab plus carboplatin plus paclitaxel (BCP), atezolizumab plus carboplatin plus paclitaxel (ACP), and atezolizumab plus bevacizumab plus carboplatin plus paclitaxel (ABCP) in chemotherapy-naive subjects who had been diagnosed with metastatic non-squamous small cell lung cancer. The trial compared: (1) ACP treatment with BCP treatment; and (2) BCP treatment with ABCP treatment. After treatment was received, the subjects were monitored for a 24 month time period. Overall Survival was tracked.

The IMpower150 study reported that Overall Survival and progression-free survival was statistically significant longer for subjects who received the combined ABCP treatment relative to subjects who received the BCP treatment. No statistically significant difference of Overall Survival was defined to be the clinical input and was therefore defined for each of the ACP and BCP treatment groups. The Overall Survival was defined as a portion of subjects in an arm remaining alive at a given point of time in the study. The denominator was normalized to account for subjects that dropped out of the study.

In this example, an integrated processing workflow is used to determine whether a subpopulation in the ACP arm can be identified where the Overall Survival of the subpopulation is statistically significant better than in the BCP arm.

VI.B. Input

Input data included variables characterizing H&E images and RNA-Seq data that includes an expression level of each of a set of genes. More specifically, a set of 41 spatial heterogeneity metrics was defined for each subject based on H&E data. The 41 spatial heterogeneity metrics included: The spatial statistics algorithms included: 5 Spatial Point Process methods (Ripley's K function features, G function features, Pair correlation function features, Mark correlation function features, and intra-tumor lymphocyte ratio); 6 Spatial Lattice Process methods (Morisita-Horn Index, Jaccard index, Sorensen index, Moran's I, Geary's C, and Getis-Ord Hotspot); and 2 Geostatistics process methods (ordinary Kriging features, indicator Kriging features).

Further, a numeric expression level was defined for each of [34,73] genes for each subject. The expression levels were normalized and log-transformed.

The input data corresponded to a baseline time slightly before treatment was initiated.

VI.C. Training Data

A training data set was defined to include 142 data elements—each corresponding to an individual subject. Each subject represented in the training data set was part of the IMpower150 study and thus, had been diagnosed with metastatic non squamous non small cell lung cancer and had not previously received chemotherapy. Each subject represented in the training data then received ACP and was monitored for 24 months.

Each data element in the training data set included the input data described in Section VI.B., a first label that indicated for how long relative to ACP treatment initiation progression-free survival was observed, and a second label that indicated for how long relative to ACP treatment initiation Overall Survival was observed. If the subject survived and did not progress for the entire 24-month time period, the first label was set to represent >24 months. If the subject survived for the entire 24-month time period, the second label was set to represent >24 months.

VI.D. Integrated Processing Workflow

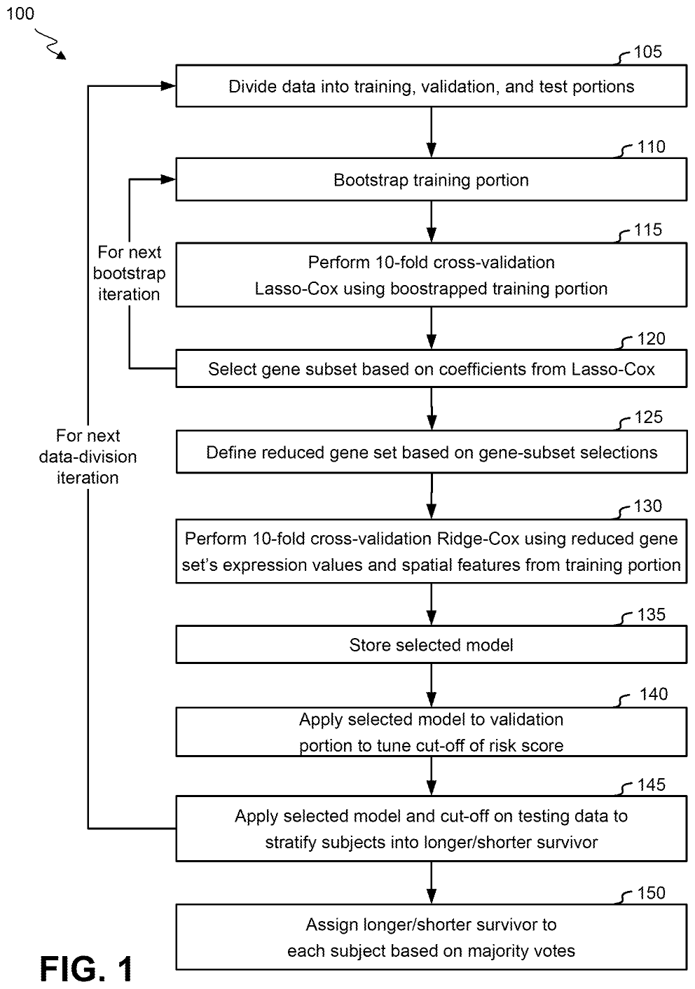

FIG. 1 illustrates a process 100 for training and using an integrated processing workflow to predict the first label and/or second label. A training data set was defined as indicated in Section VI.C. A bootstrap technique was performed to reduce a variable set.

More specifically, at block 105, the training data set was split into training, validation, and test portions. The training portion included 60% of the data elements, the validation portion included 20% of the data elements, and the test portion included 20% of the data elements. The split was performed using a pseudo-random technique.

Blocks 110-125 include actions performed by a variable-focus model to shrink a number of gene-expression variables. At block 110, the training portion was bootstrapped into multiple resamples. In this example, the bootstrapping was performed 1,000 times. For each of these resamples, at block 115, a Lasso-Cox model was used to perform a 10-fold cross-validation. The Lasso model is a regression model, though a constraint is imposed that requires a sum of coefficients to be less than a threshold. This constraint can result in the coefficients of multiple input variables being set to zero. The constraint is controlled by a hyperparameter λ. Larger values of λ result in more coefficients being shrunk to zero and less variables being included in a reduced variable set. An R function was used to select its own sequence of λ and find an optimal λ values via an internal cross-validation.

One complication with the IMpower150 is that the data indicates whether individual subjects survived (and/or progressed) over a 24-month period, but a number of subjects tracked declined throughout the time period (due to subjects leaving the study or not surviving). A Cox Proportional Hazards Model (with L1 regularization and stability selection) was thus used to account for the change in sample size.

Specifically, the Lasso model was selected as a regularization technique, and the Cox proportional hazard model was used as a loss function to select genes using a permutation strategy with a false discovery rate less than 0.001. An output produced by the Lasso-Cox model included a coefficient for each of the gene-expression variables. Many of the coefficients were zero. Therefore, at block 120, a subset of the set genes represented by the gene-expression variables was then defined to include the genes corresponding to a non-zero coefficient.

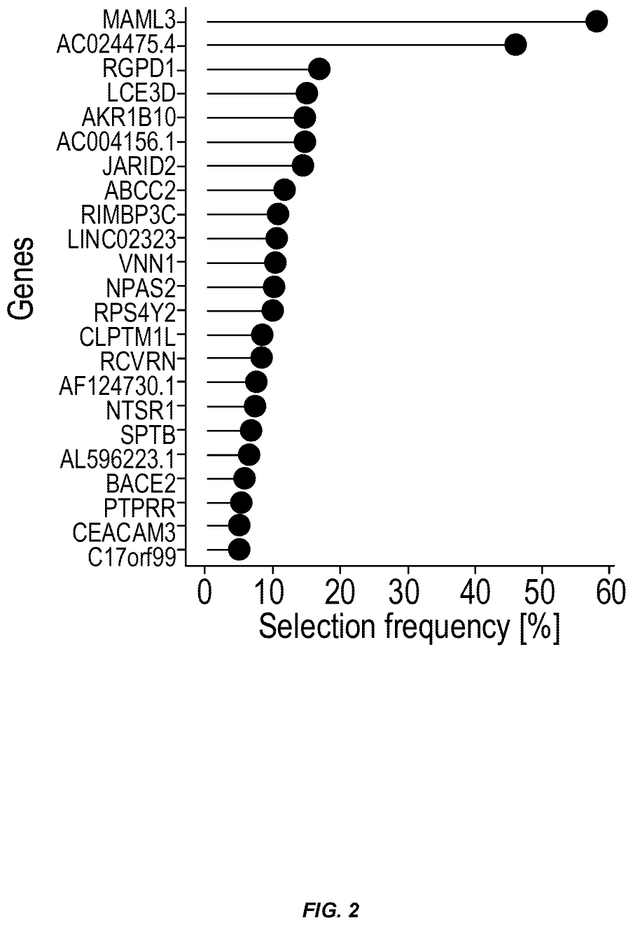

Blocks 110-120 were repeated 1,000 times. Each time, the training portion was bootstrapped differently, such that different data was assessed using the Lasso-Cox model at block 115. FIG. 2 shows how frequently each of an exemplary set of genes were selected to be in the subset of genes.

After all bootstrapping iterations were completed, process 100 continued to block 125, where a reduced gene set was defined based on the gene subsets selected during the bootstrapping iterations. Specifically, for each gene in the gene set, a count identified a number of times that the gene was included in a gene subset, and genes that were associated with the seven highest counts were included in the reduced gene set.

At block 130, another 10-fold cross-validation was performed using a Ridge-Cox model to determine parameter values of a data integration Ridge model. This data integration Ridge model not only receives expression values from the reduced gene set but also the 41 spatial heterogeneity metrics from the training elements. Training the Ridge-Cox model resulted in learning a coefficient for each gene in the reduced gene set and a coefficient of each of the spatial heterogeneity metrics.

At block 135, a selected model was stored. The selected model included a particular coefficient for each gene in the reduced gene set and for each spatial heterogeneity metric, where the particular coefficient was selected using the 10-fold cross validation performed at block 130. The selected model was configured to predict a risk score of occurrence of progressing and/or not surviving.

At block 140, the selected model was applied to the validation portion of the training data set to tune a cut-off of the risk score. The cut-off was used to discriminate between subjects predicted to survive a treatment versus not surviving. In order to determine a cut-off, multiple cut-offs were assessed (by varying the cut-off from 0.2 to 0.8 in increments of 0.05) based on an accuracy of predicting Overall Survival. The cut-off corresponding to the highest accuracy was selected.

At block 145, the selected model and cut-off were used to process data for each subject represented in the testing data. The selected model generated a risk score, and the cut-off was used to predict whether the risk score indicated that the subject will survive for a longer duration (e.g., more than 24 months) or a shorter duration. An interim assignment to a longer survivor or shorter survivor class was assigned based on the prediction.

Blocks 105-145 were repeated 1,000 times using different data-division iterations. Each time, a different subset of subjects were represented in the testing data. However, most subjects were represented in the testing data many times.

At block 150, for each subject, the subject was assigned to a longer-survivor class or a shorter-survivor class based on the interim predictions from the data-division iterations. Specifically, each assignment was selected based on the majority interim class assignment across the data-division iterations.

It will be appreciated that, due to the bootstrapping implementation, multiple data integration models were defined for this Example, and a post-processing majority-votes analysis was used (at block 150 to select a class assignment) to process results from the various models.

VI.E. Single-Modality Processing Workflows

Process 100 uses both gene-expression data and spatial heterogeneity metrics (corresponding to H&E data). These data types were also separately analyzed to determine the predictive power of each modality separately. Specifically, for each of 1,000 training/validation/test partitions, gene-expression training data was processed using blocks 105-125 of process 100, and model parameters were generated based on the bootstrap iterations' coefficients. These coefficients were then collectively assessed to . . . . A subpopulation was defined to include subjects who had received the ACP treatment and . . . . Similarly, for the spatial heterogeneity metrics . . . .

VI.F. Results

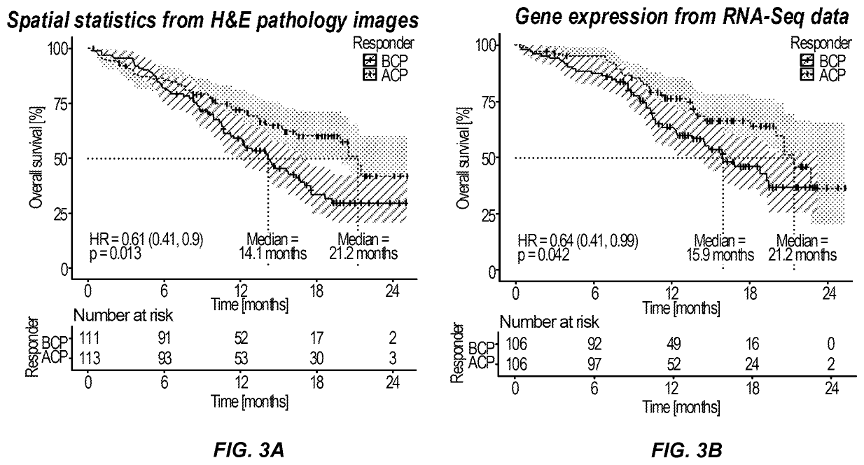

FIGS. 3A and 3B show Overall Survival curves for subjects who received the BCP treatment and for a subpopulation of subjects who received the ACP treatment, where the subpopulation was selected using the single-modality models. Specifically, FIG. 3A shows survival cures for the BCP subjects relative to subjects predicted to be long survivors using H&E data, and FIG. 3B shows survival cures for the BCP subjects relative to subjects predicted to be long survivors using gene-expression data. (FIGS. 3A and 3B also show number at risk statistics, which were the number of alive subjects remaining in the study at each time point who are at risk of having an event in the future.) The Hazard ratio based on survival data for the BCP-treatment subject population relative to the survival data of the ACP-treatment subpopulation selected using the H&E data was 0.61 (with a confidence interval of [0.41, 0.90]). The Hazard ratio based on survival data for the BCP-treatment subject population relative to the survival data of the ACP-treatment subpopulation selected using the digital pathology data was 0.64 (with a confidence interval of 0.41, 0.99]). With respect to both analyzes, the subpopulation in the ACP arm had a significantly lower risk of death during the monitoring period relative to the BCP arm (p=0.013 when using H&E data for defining the subpopulation and p=0.042 when using digital pathology data for defining the subpopulation).

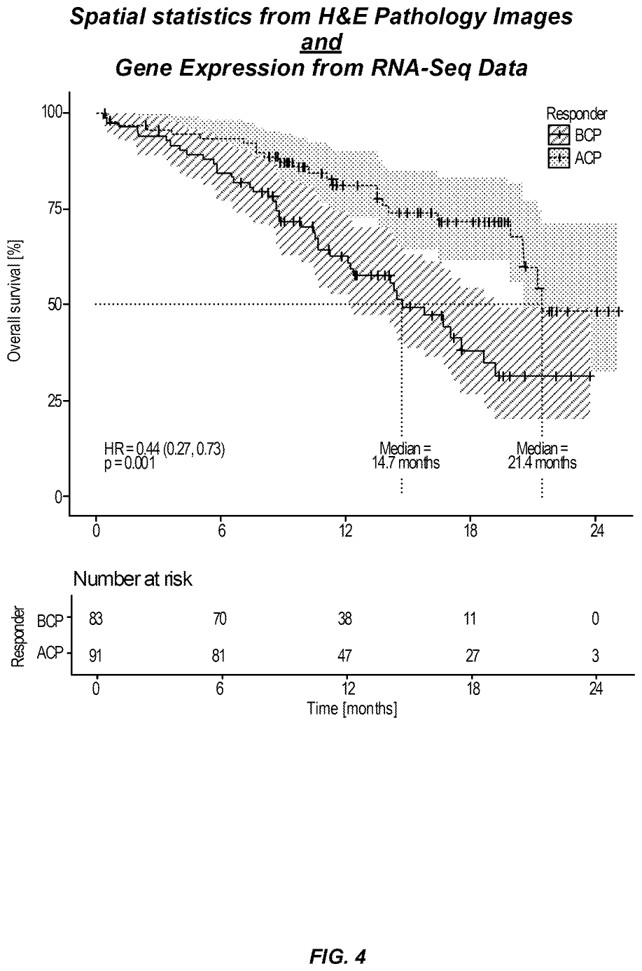

FIG. 4 shows the Overall Survival curves for subjects who received the BCP treatment and for a subpopulation of subjects who received the ACP treatment, where the subpopulation was selected using the multi-modality approach described in Section VI.D. The survival statistics of this subpopulation were higher than those corresponding to subpopulations calculated using a single-modality approach. For example, the number of subjects in the ACP subpopulation deemed to be at risk of dying each of 0, 6, and 12 months after initiation of treatment were lower when the subpopulation was defined based both on H&E data and gene-expression data, relative to either data type alone. Further, the Hazard ratio based on survival data for the BCP-treatment subject population relative to the survival data of the ACP-treatment subpopulation was lower when the subpopulation was selected using both H&E and digital pathology data (0.44; confidence interval=[0.27, 0.73]) relative to the single-modality Hazard ratios (0.61 and 0.64). The significance of the difference between the survival curves when both data types were used to select the ACP-treatment subpopulation (p=0.001) was also an order of magnitude lower than the significance with only one modality was used to define the subpopulation. Data-driven selection of genes relevant for the response prediction included MAML3, AC024475.4, RGPD1, LCE3D and AC004156.1. Notably, increased MAML3 expression as a transcriptional coactivator of Notch, might be involved in driving the cancer stem cell state and thus impairing drug effectiveness.

VI.G. Interpretation

These results show that survival prediction is significantly improved when it is based on both gene-expression data and digital pathology data, relative to either data-type individually. Thus, an integrated processing workflow may be useful in predicting whether a given subject will respond to a particular treatment.

Further, retrospective analysis identified an extent to which various genes were selected for inclusion in gene-expression input data. Expression levels of genes that were frequently selected (e.g., one or more genes shown in FIG. 2 and/or one or more genes listed in Section VI.F, such as MAML3) may be used as a biomarker for predicted responsiveness, and/or genes that were frequently selected may be assessed in an assay to predict responsiveness to the particular treatment. Thus, these results may inform development of a companion diagnostic that predicts individualized treatment response.

### VII. Additional Considerations

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification, and variation of the concepts herein disclosed may be resorted to by those skilled in the art, and that such modifications and variations are considered to be within the scope of this invention as defined by the appended claims.

The description provides preferred exemplary embodiments only, and is not intended to limit the scope, applicability or configuration of the disclosure. Rather, the description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

1. A computer-implemented method comprising:
accessing a first data set corresponding to one or more digital pathology images and to a particular subject;
accessing a second data set corresponding to expression levels of a set of genes and to the particular subject; and
generating a result that corresponds to a predicted current state of a medical condition or to a predicted progression of the medical condition, the result being generated by processing the first data set and the second data set using a machine-learning model.

2. The computer-implemented method of claim 1, further comprising:
generating the second data set by filtering an initial set of expression levels of a larger set of genes, wherein the set of genes were identified using a variable-focus model configured to reduce an input data set.

3. The computer-implemented method of claim 1, wherein the first data set includes a set of spatial heterogeneity metrics identified by:
detecting depictions of a set of immune cells in the one or more digital pathology images;
detecting depictions of a set of tumor cells in the one or more digital pathology images;
generating each of the set of spatial heterogeneity metrics based on locations of the depictions of the set of immune cells and based on locations of the set of tumor cells.

4. The computer-implemented method of claim 1, further comprising:
generating the first data set by processing the one or more digital pathology images using a first upstream machine-learning model, the first data set including a first preliminary result corresponding to a first preliminary prediction of the current state or to a first preliminary prediction of the medical condition.

5. The computer-implemented method of claim 1, further comprising:
generating the second data set by processing the expression levels of the set of genes using a second upstream machine-learning model, the second data set including a second preliminary result corresponding to a second preliminary prediction of the current state or to a second preliminary prediction of the medical condition.

6. The computer-implemented method of claim 1, wherein the result corresponds to the predicted progression of the medical condition assuming that the particular subject receives a particular treatment and/or includes a probability of survival.

7. The computer-implemented method of claim 6, further comprising:
generating another result that corresponds to a another predicted progression of the medical condition assuming that the particular subject receives another particular treatment, the other result being generated by processing the first data set and the second data set using another machine-learning model; and
selecting one of the particular treatment or other particular treatment to treat the particular subject or to recommend for treatment of the particular subject.

8. The computer-implemented method of claim 1, wherein the medical condition includes a particular type of cancer.

9. The computer-implemented method of claim 1, further comprising: determining whether the particular subject is eligible to participate in a clinical study based at least in part on the result.

10. The computer-implemented method of claim 1, further comprising: selecting a treatment arm to which the particular subject is to be assigned based at least in part on the result.

11. A computer-implemented method comprising:
accessing a data set corresponding to one or more digital pathology images and to a particular subject; and
generating one or more predicted gene-expression levels by processing the data set using a machine-learning model.

12. The computer-implemented method of claim 11, wherein the data set includes a set of spatial heterogeneity metrics identified by:
detecting depictions of a set of immune cells in the one or more digital pathology images;
detecting depictions of a set of tumor cells in the one or more digital pathology images;
generating each of the set of spatial heterogeneity metrics based on locations of the depictions of the set of immune cells and based on locations of the set of tumor cells.

13. A computer-implemented method comprising:
accessing a set of training data elements, each of the set of training data elements corresponding to an individual subject diagnosed with a medical condition and including:
a first data set corresponding to one or more digital pathology images;
a second data set corresponding to expression levels of a set of genes; and
a label that indicates a state of the medical condition of the individual subject or a progression of the medical condition observed subsequent to a time point associated with collection of the one or more digital pathology images and to time point associated with collection of the expression levels;
training a machine-learning model using the training data elements, wherein values for a set of parameters are learned during the training; and
determining a data signature for a particular type of state of the medical condition or a particular type of progression of the medical condition using at least one of the values for the set of parameters, wherein the data signature includes a value or range for an expression level each of at least some of the set of genes and a value or range for each of one or more digital pathology metrics.

14. The computer-implemented method of claim 13, further comprising:
accessing a subject-specific data element corresponding to a particular subject diagnosed with the medical condition and not represented in the set of training data elements, the subject-specific data element including a subject-specific value for each of the at least some of the set of genes and for each of the one or more digital pathology metrics;
generating a result corresponding to a predicted state of the medical condition of the particular subject or predicted progression of the medical condition of the particular subject, the result being generated by processing the subject-specific data element using the trained machine-learning model; and
outputting the result, at least some of the subject-specific values in the subject-specific data set, and the data signature.
